(19) United States
(12) Patent Application Publication
(10) Pub. No.: US 2001/0036938 A1
(43) Pub. Date: Nov. 1, 2001

(54) Use of oral beclomethasone dipropionate to treat liver inflammation

(76) Inventor: George B. McDonald, Bellevue, WA (US)

Correspondence Address: LYON & LYON LLP, 633 WEST FIFTH STREET, SUITE 4700, LOS ANGELES, CA, 90071, US

(21) Appl. No.: 09/753,852

(22) Filed: Jan. 3, 2001

Related U.S. Application Data
(60) Provisional application No. 60/178,782, filed on Jan. 28, 2000.

(51) Int. Cl. A61K 31/573

(52) U.S. Cl. 514/179

(57) ABSTRACT

Liver inflammation caused by liver disease is treated orally with biclomethasone dipropionate or a metabolite thereof.

### FIELD OF THE INVENTION

This invention relates to the treatment of liver inflammation and more particularly to the treatment of liver inflammation by an orally effective therapeutic agent.

### BACKGROUND OF THE INVENTION

There are a number of liver diseases characterized by inflammation of the liver, that is, infiltration of the liver with lymphocytes, plasma cells, and neutrophils. These inflammatory cells accumulate in the liver in response to numerous stimuli, for example, ethanol-induced liver injury (alcoholic hepatitis), fatty liver (non-alcoholic steatohepatitis), viral infection (chronic hepatitis B or C), medications (drug-liver injury), as well as in diseases whose cause or stimulus to inflammation is unknown, for example, primary biliary cirrhosis, sclerosing cholangitis, and autoimmune hepatitis. There is evidence in many of these disorders that the damage to the liver is not as much related to the initial insult or primary cause as to the inflammatory response. There is evidence that inflammatory cells cause damage to liver cells (hepatocytes) and also stimulate other cells in the liver, for example, Kupffer cells (derived from monocyte-macrophage cells) and stellate cells (a form of myofibroblast). For example, the development of fibrosis (scarring) in the liver that can lead to cirrhosis of the liver results from the constant stimulus of stellate cells by proteins released by inflammatory cells and Kupffer cells that lead to production of collagen by the affected stellate cells. Treatment of inflammatory diseases of the liver can be directed at either the primary stimulus, when one is known, or at the inflammatory process or at the actions of Kupffer cells and stellate cells. Examples of treatment of the primary stimulus to liver inflammation would be cessation of alcohol consumption by patients who have alcoholic hepatitis and the treatment of hepatitis B or hepatitis C with antiviral drugs. However, these treatments are not always effective. For example, the ability to eliminate hepatitis C virus from the liver with the best currently available antiviral therapy is about 50% and sometimes the inflammatory liver disease persists even when the original stimulus has been eliminated, for example, alcoholic hepatitis. The other disadvantage to treatments which seek to treat the inflammatory response is that the medications used for this purpose, corticosteroids such as prednisone, antimetabolites such as methotrexate, alkylating agents such as cyclophosphamide and azathioprine, is that they cause a generalized immunosuppression throughout the body, and their side effects occur throughout the body. While these treatments may be effective in suppressing liver inflammation, they are limited by side effects because they are not liver-specific. For most other inflammatory liver diseases where the primary stinumus is not known, there are no known effective treatments.

### SUMMARY OF THE INVENTION

According to this invention, liver diseases characterized by inflammation of the liver can be effectively treated by oral administration of beclomethasone dipropionate (BDP) or a metabolite thereof to a patient having such a liver disease. BDP or a metabolite thereof is administered orally to a patient suffering from a liver disease characterized by inflammation of the liver in any therapeutically effective dosage amount, generally in an amount of from about 2 mg/day to about 12 mg/day in any form suitable for oral administration, such as capsules, pills or emulsions. If desired, other agents may be included in such oral formulations.

### DETAILED DESCRIPTION OF THE INVENTION

Beclomethasone dipropionate (BDP) is a compound available from a number of commercial sources, such as Schering-Plough Corporation of Kenilworth, N.J. in bulk crystalline form and has the following structure (i.e., beclomethasone 17,21-dipropionate:

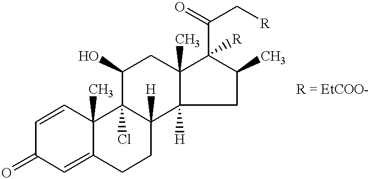

In the practice of this invention, a therapeutically effective amount of a BDP is administered to a patient in need thereof. In general terms, a therapeutically effective amount of a BDP is an amount which, when delivered orally, treats the inflammation of the liver associated with diseases of the liver. Such an amount may be readily determined by one skilled in the art by well-known dose-response investigations, and will generally range from 2 mg/day to 12 mg/day, and more typically range from 4 mg/day to 8 mg/day.

Liver blood flow comes from two sources, the hepatic artery, which receives its blood supply from the aorta, which receives blood from the left ventricle of the heart, and the portal vein, which is the venous blood supply that comes from the intestinal tract and the spleen. The liver is thus unique in the body in receiving this dual blood supply. Blood from these two sources mix when they reach the sinusoids of the liver, that is, the channels that transport blood from the portal areas to the central veins. These sinusoids are lined with a specialized type of endothelium that is fenestrated, allowing the plasma component of blood and medications carried in the plasma component to enter the space of Disse and thereby bathe both the liver cells and the other cells in the liver lobule, such as inflammatory cells, Kupffer cells, and stellate cells. When medications are taken orally and delivered into the intestinal tract, they have two routes of transport into the body: One is via the portal vein to the liver, and the other is via intestinal lymphatics to the cysterna chyli and thoracic duct into the venous circulation, avoiding passage through the liver. The partitioning of medications between the portal circulation and the lymphatics is a complex one, but in general, lipophilic (fat-loving) substances are generally transported via the lymphatics, and non-or less-lipophilic substances through the portal venous system to the liver. Oral BDP and similar corticosteroid medications are transported into the portal circulation either as the parent molecule or as a metabolite. For example, BDP is partially metabolized in the intestinal mucosa to beclomethasone monopropionate (BMP), which is a highly active anti-inflammatory molecule. After an oral dose of BDP is delivered to the intestine, both BDP and BMP are delivered to the liver in the portal circulation in pharmacological amounts. The rationale for the use of oral BDP for inflammatory diseases of the liver is therefore that (1) adequate amounts of active BDP and BMP reach the portal areas, sinusoids, and space of Disse in the liver; (2) these are the sites of inflammatory cells in the liver in patients with inflammatory liver diseases; (3) liver cells are capable of metabolism of BDP and BMP, such that the blood that exits the liver via the hapatic vein has little BDP or BMP in it as it returns to the heart; (4) inflammatory cells outside the liver are not exposed to large amounts of BDP or BMP, and thus the systemic side effects related to immune suppression are avoided; and (5) other organs and tissues in the body are not exposed to large amounts of BDP or BMP, and thus, side effects related to bone loss, carbohydrate metabolism, lipid metabolism, and other side effects, are minimized.

There are numerous permutations and combinations of formulations and dosing of BDP that might be proposed in this application. For example, if high-dose pulse therapy, where liver inflammatory cells were exposed to high levels of BDP and BMP for a short period of time, were desirable, then non-enteric coated formulation of BDP might be used, where all of the dose is released in the stomach, delivered to the duodenum with the liquid phase of gastric emptying, and metabolized and transported into the portal circulation over a short period of time. This sort of dosing might be done infrequently, to effect the desired anti-inflammatory activities in the liver but minimizing the amount of BDP metabolite in the general circulation, thus minimizing exposure of tissues other than the liver to BDP metabolites.

If one wanted to avoid exposing the upper intestinal tract to BDP therapy because of its affect on local mucosal immunity, one might deliver all of the BDP in enteric coated capsules that would be delivered to the duodenum in the interdigestive phase of gastric emptying, dissolve in the alkaline environment of the small intestine, and be metabolized and transported by the mid- to distal intestine over a short period of time. This sort of dosing might be done infrequently, to effect the desired anti-inflammatory activities in the liver but minimizing the amount of BDP metabolite in the general circulation, thus minimizing exposure of tissues other than the liver to BDP metabolites.

If one wanted to provide a constant but low level exposure of BDP and BMP to the liver, formulations of BDP could be devised that would release BDP over an extended period of time. This approach could use the technology of the Alza Inc. where different polymer coatings extend the release time of one orally delivered dose that would be delivered to the duodenum in the interdigestive phase of gastric emptying. This sort of dosing could allow very low levels of BDP and BMP to be delivered to the liver per unit of time, but continuously, over a 24-hour period. This approach could allow more thorough metabolism of BDP metabolites as they pass through the liver in low concentration, enough to effect suppression of inflammation but not enough to gain access to the systemic circulation.

With the foregoing description of the invention, those skilled in the art will appreciate that modifications may be made to the invention without departing from the spirit thereof. Therefore, it is not intended that the scope of the invention be limited to the specific embodiments illustrated and described.

I claim:

1. A method of treating a patient having a liver disease characterized by inflammation of the liver comprising orally administering to said patient a therapeutically effective amount of beclomethasone dipropionate or a metabolite thereof.

2. A method according to claim 1 wherein beclomethasone dipropionate is orally administered to the patient in a dosage of from about 2 mg/day to about 12 mg/day.

3. A method according to claim 2 wherein the beclomethasone dipropionate is orally administered to the patient in the form of a capsule, pill or emulsion.
